(19) United States
(12) Patent Application Publication
(10) Pub. No.: US 2026/0256919 A1
(43) Pub. Date: Sep. 3, 2026

(54) BIOENHANCERS

(71) Applicant: aXichem AB, Malmö (SE)

(72) Inventors: Torsten HELSING, Kleppestø (NO); Lucas ALTEPOST, Nesøya (NO); Bomi FRAMROZE, Menlo Park, CA (US); Erik LAGER, Lund (SE)

(73) Assignee: aXichem AB, Malmö (SE)

(21) Appl. No.: 18/838,380

(22) PCT Filed: Feb. 17, 2023
(86) PCT No.: PCT/EP2023/054021

§ 371 Date: Aug. 14, 2024

(30) Foreign Application Priority Data
Feb. 18, 2022 (NO) 20220225

(51) Int. Cl. A61K 47/18; A61K 9/00; A61K 31/196; A61K 31/381; A61K 31/4045; A61K 31/433; A61K 31/522

(52) U.S. Cl. CPC A61K 47/18; A61K 9/0053; A61K 31/196; A61K 31/381; A61K 31/4045; A61K 31/433; A61K 31/522

(57) ABSTRACT

The present invention relates to synthetic capsaicyns, tautomers or salts thereof and their use as bioenhancers. More particularly, the invention provides synthetic capsaicyns acting as inhibitors of certain Cytochrome P450 (CYP) isoforms. Further, the invention relates to compositions comprising capsaicyns for use as bioenhancers on active substances metabolized by CYP1A2 and CYP2D6. Such compositions or compounds may preferably be for oral administration of pharmaceuticals, nutraceuticals and supplements, increasing the bioavailability of the active substances.

### FIELD OF THE INVENTION

The present invention relates to synthetic capsaicyns, tautomers or salts thereof and their use as bioenhancers. More particularly, the invention provides synthetic capsaicyns acting as inhibitors of certain Cytochrome P450 (CYP) isoforms. Further, the invention relates to compositions comprising compounds for use as bioenhancers of active substances. Such compositions or compounds may preferably be for oral administration of pharmaceuticals, nutraceuticals and supplements.

### BACKGROUND OF THE INVENTION

Absorption of substances and metabolism of said substances affect the amount of substance available in the body. Poorly absorbed substances require a higher administration of said substance or other adjustments to the administration regime to exert an effect in the body than if said substance were better absorbed. Poorly absorbed substances have a low bioavailability.

To obtain a therapeutic effect of a substance, such as a drug, the substance must be well absorbed and remain unmetabolized, thereby increasing bioavailability which is the key to making an enhancement that delivers proven benefits. Whether a drug substance is administered into the human body through the oral, intravenous or parenteral route, they will be metabolized by the hepatic portal system (liver) which is the site for most metabolism, by metabolizing enzymes, which will degrade the substances delivered for easy removal from the body. In addition to the route of administration, several other factors affect bioavailability, including the state of the drug.

There are several drawbacks for substances with poor bioavailability. A higher amount of the substance is needed to achieve the desired effect, e.g., by increasing the dose. This may result in the appearance of unwanted side effects. An unreasonable amount of substance may be required, making it difficult or unrealistic for ingesting the substance, causing reduced compliance. A further drawback is the potential of an extensive burden on raw materials and supplies. The raw materials may be scarce, and their removal may have ecological disadvantages. The supply is subject to environmental fluctuations causing an unreliable raw material supply.

Increased ingestion of a substance increases the excretion amounts of the substances and/or metabolites thereof. This can cause greater environmental pollution if said substances and/or metabolites have toxic effects on the environment, potentially including wildlife.

Furthermore, higher costs are associated with an increased amount of required substance. Unreasonably high costs may present a barrier for those that are not financially strong, creating a divide in society such as between those who can afford medications and those who cannot afford medications and thus experience undue disease burden and/or mortality.

Bioenhancers are agents increasing the bioavailability of other substances. Piperine was validated as a bioenhancer in 1979. From then, a variety of bioenhancers have been discovered. Some examples of known bioenhancers are: curcumin, piperine, quercetin, gingerols, allicin, glycyrrhizin, genistein, sinomenine, Stevia rebaudiana, Aloe vera, lysergol, Carum carvi, niaziridin, capsaicin, naringin, Zingiber officinale, Ammannia multiflora, capmul, cow urine distillate. Several of these bioenhancers are described in Dudhatra et al. (2012) The Scientific World Journal, doi: 10.1100/2012/637953.

Several of these bioenhancers must be extracted from natural ingredients. This can be an inefficient process with a low yield and/or with the presence of unwanted substances. The product may contain different isomeric forms with varying chemical properties and thus variable degrees of suitability for the purpose as bioenhancer. The degree of purity is therefore variable. Consequently, there is a need for new and alternative bioenhancers with high purity that may be produced synthetically.

WO2019/077115 of the applicant reports that capsaicyns can be used as a bioenhancer in pharmaceutical compositions, nutraceutical compositions, supplemental compositions, or in compositions for inclusion in feed or food. Particularly, WO2019/077115 suggests including capsaicyns in feed, as a bioenhancer.

The object of the present invention is to provide bioenhancers that may increase the bioavailability of specific active substances for use in nutraceuticals, supplements or pharmaceuticals.

### BRIEF SUMMARY OF THE INVENTION

The inventors have discovered that synthetic capsaicyns may act as direct inhibitors of certain Cytochrome P450 (CYP) isoforms.

Surprisingly, the applicant has found that the R-capsaicyns according to Formula (I) can effectively be used as, and act as, bioenhancers of substrates of the Cytochrome P450 Isoform 1A2 (CYP1A2) and Cytochrome P450 Isoform 2D6 (CYP2D6).

In one aspect, the invention provides a compound of Formula (I)

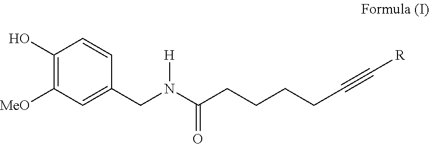

wherein R is alkyl, trifluoromethyl, cycloalkyl, phenyl, or halogen, for use as a bioenhancer of substances metabolized by either of CYP1A2 and CY2D6.

In another aspect, the invention provides a composition comprising
i) at least one compound of Formula (I); and
ii) at least one active substance, tautomers or salts thereof selected from the group of CYP1A2 substances and CYP2D6 substances.

The compound of formula (I) has a bioenhancing effect on the at least one active substances or tautomers or salt thereof.

Another aspect is a composition comprising a compound of Formula (I), for use as a bioenhancer of at least one active substance selected from CYP1A2 substances and

CYP2D6 substances.

A further aspect is the use of at least one compound of Formula (I) as a bioenhancer of a at least one active substance selected from the group of CYP1A2 and CYP2D6 substances. And further, the invention provides the use of a composition comprising at least one compound of Formula (I), as a bioenhancer of at least one active substance selected from the group of CYP1A2 and CYP2D6 substances.

Further, the invention provides a composition for use in treatment or prevention of a condition, disorder or a disease comprising a compound of Formula (I), wherein the compound of Formula (I) act as a bioenhancer of at least one active substance selected from CYP1A2 substances and CYP2D6 substances used in the treatment or prevention.

A further aspect is a method comprising administering an effective amount of at least one compound of Formula (I); wherein the compound of Formula (I) act as a bioenhancer of at least one active substance selected from CYP1A2 substances and CYP2D6 substances used in the treatment or prevention.

Also provided is a method for increasing the bioavailability of an active substance in a subject, wherein the active substance is selected from CYP1A2 substances and CYP2D6 substances, the method comprises a step of administering a compound of Formula (I) to the subject, the compound acting as a bioenhancer of the at least one active substance.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

By “treatment” and “treating”, we mean therapeutic applications in response to at least one existing condition, disorder or disease for humans.

By “prevention” and “preventing”, we mean prophylactic use and/or vaccination as preventative measures against at least one condition, disorder or disease.

By “condition”, “disorder” or “disease”, we mean physical and/or mental changes from and/or disturbances of a regular physiological and/or mental state.

By CYP1A2, we mean Cytochrome P450 Isoform 1A2. By CYP2D6, we mean Cytochrome P450 Isoform 2D6.

By CYP1A2 substances, we mean CYP1A2 substrates, being substances that are metabolized by CYP1A2.

By CYP2D6 substances we mean CYP2D6 substrates, being substances that are metabolized by CYP2D6.

The invention relates to at least one chemical compound of Formula (I)

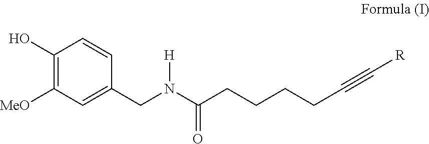

wherein R is alkyl, trifluoromethyl, cycloalkyl, phenyl, or halogen.

When said substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl.

When said R comprises a carbon chain, the carbon chain may preferably be alkyl. The carbon chain may have a 1 to 6 carbon atom long chain, more preferably a 1 to 4 carbon atom long chain. More preferably, R may be isopropyl or a C4 alkyl. R may also preferably be phenyl. The compound wherein R is phenyl is thus termed phenylcapsaicyn.

In one embodiment, the compound of Formula I is selected from the group of methylcapsaicyn, ethylcapsaicyn, a propylcapsaicyn, a butylcapsaicyn, and phenylcapsaicyn.

The compound of Formula (I) is herewith termed R-capsaicyn. It is important to note the difference in structure between capsaicin, a pepper analogue, and R-capsaicyns. Firstly, capsaicin contains a double bond instead of a triple bond, which is present in R-capsaicyn. Hence, the difference in the endings -in and -yn. Said substituent R is therefore not a substituent at the end of a capsaicin molecule, but at the end of an analogue, which may have different properties from capsaicin. EP patent 1 670 310 B1 of avoiding the limitations and disadvantages of extracting the compounds from natural products and raw materials.

The invention provides a compound of Formula (I) for use as a bioenhancer of certain substances. A bioenhancer is an agent utilised to increase the bioavailability of at least one particular substance. The skilled person is familiar with the term “bioavailability” as to indicate the fraction (F) of an administered substance that reaches the systemic circulation as an intact substance. F is a measure by comparison of plasma substance concentration versus time by the chosen route of administration compared to plasma substance concentration versus time by intravenous administration. The areas under the plasma concentration curves (AUC) estimates F given by the Formula AUC chosen route/AUC intravenous. The numerical value of F is between 0 and 1, wherein 0 is no bioavailability and 1 is bioavailability at the level of intravenous administration. A bioenhancer will therefore increase F towards 1. The value of F may also be given as a percentage. The skilled person is familiar with performing such measurements.

The ability for a compound to act as a bioenhancer can be due to a variety of physiological mechanisms. One mechanism includes the inhibition of endogenous enzymes degrading the material delivered and taken up through the gut. Cytochrome P450 enzymes (CYPs) are endogenous enzymes involved in hepatic metabolism of a majority of the pharmaceuticals in use. By inhibiting such isozymes, metabolism of certain pharmaceuticals is slowed down or prevented. This may prolong the half-life of the pharmaceutical. If those pharmaceuticals are in their active form, they can exert their activity for a longer duration and they will be in a higher concentration compared to situations where those isozymes are not inhibited. In WO2019/077115 of the applicant it was disclosed that phenylcapsaicyn may act as a bioenhancer through exerting inhibitory effects on cytochrome P450 enzymes. However, the CYP family of enzymes includes a vast group of different enzymes, having different roles in drug metabolism. The applicant has sought to better understand how the findings reported in WO2019/077115, that phenylcapsaicyn may act as a bioenhancer inhibiting cytochrome P450 enzymes, can be used specifically and advantageously.

The current invention is partly based on findings from studies performed by the applicant. One such study, as further detailed in Example 1, provides an evaluation of the direct inhibitory potential of phenylcapsaicyn, a compound of Formula (I), against seven human Cytochrome P450 isoforms using human liver microsomes. The results of the study show that phenylcapsaicyn is a direct inhibitor of CYP1A2 and CYP2D6. The inhibitory effect of the compound supports an expected drug bio enhancing effect with concomitantly administered substances that are metabolized by either of CYP1A2 and CYP2D6. Surprisingly, the inventors have hence found that compounds of Formula (I) inhibit CYP1A2 and CYP2D6, herein called CYP1A2 and CYP2D6 inhibitory compounds.

The invention provides compounds of Formula (I) as bioenhancers of substances metabolized by either of the CYP1A2 and CY2D6 enzymes. The bioenhancers of Formula (I) have been found to increase the bioavailability of substances metabolized by either of CYP1A2 and CYP2D6.

In one aspect, the invention provides a compound of Formula (I) for use as a bioenhancer of substances metabolized by either of the CYP1A2 and CY2D6 enzymes. Similarly, the invention provides the use of a compound of Formula (I) as bioenhancer of substances metabolized by either of the CYP1A2 and CY2D6 enzymes. Hence, the compound of Formula (I) may be used as a bioenhancer for at least one active substance selected from CYP1A2 substances and CYP2D6 substances to increase the bioavailability of the at least one substance.

In another aspect, the invention provides a composition comprising at least one compound of Formula (I), wherein the at least one compound of Formula (I) has a bioenhancing effect on CYP1A2 substances and CYP2D6 substances. In one embodiment, the composition comprises
i) at least one compound of Formula (I); and
ii) at least one active substance, tautomers or salts thereof selected from the group of CYP1A2 substances and CYP2D6 substances.

Hence, the invention provides a composition comprising at least one compound of Formula (I), and such composition for use as a bioenhancer of at least one active substance selected from CYP1A2 substances and CYP2D6 substances.

In one embodiment, the composition of the invention may comprise a plurality of compounds of Formula (I) (bioenhancers), such as a mix of two or more different compounds of Formula (I). Wherein a plurality of compounds are present, preferably all of these have a bioenhancing effect on the at least one active substance, tautomers or salts thereof. The plurality of compounds of Formula (I) may have bioenhancing effects on the same, similar or different of the at least one active substance, tautomers or salts thereof. In other words, the composition may comprise two or more bioenhancers acting on an active substance. It may comprise two or more bioenhancers acting on two or more active substances. The two or more bioenhancers may act on the same active substance(s), different active substance(s) or on overlapping active substance(s).

In one embodiment, the composition of the invention comprises at least one active substance, tautomers or salts thereof selected from the group of CYP1A2 substances and CYP2D6 substances. The compound of Formula (I) has a bioenhancing effect on the at least one active substance, tautomers or salts thereof. The bioenhancer and the at least one active substance, tautomers or salts thereof, may be provided as one formulated composition, or the two may form separate sub-compositions of the claimed composition, as further detailed below.

Hence, the invention provides a composition comprising
i) the R-capsaicyn inhibitor of Formula (I); and
ii) at least one active substance, tautomers or salts thereof selected from the group of CYP1A2 substances and CYP2D6 substances, wherein the active substance(s) is provided either as a separate composition to i) or the two components i) and ii) form, such as are mixed into, one composition formulation. The composition is for use as a bioenhancer of the at least one active substance selected from CYP1A2 substances and CYP2D6 substances.

Hence, the invention provides a combined composition for simultaneous, separate or sequential use, said composition comprising
i) a first composition comprising the R-capsaicyn inhibitor of Formula (I);
ii) a second composition comprising at least one active substance, tautomers or salts thereof selected from the group of CYP1A2 substances and CYP2D6 substances. Where the two compositions of the combined composition are to be administered simultaneously, they may be mixed, and potentially formulated, into one composition.

In one embodiment, the bioenhancer and the at least one active substance are provided as one formulation. Accordingly, the invention provides a composition comprising
i) at least one compound of Formula (I); and
ii) at least one active substance, tautomers or salts thereof selected from the group of CYP1A2 substances and CYP2D6 substances. In this embodiment, the two components i) and ii) may be mixed into one formulation comprising both the at least one compound of Formula (I), and the at least one active CYP1A2 or CYP2D6 substance, tautomer or salt thereof. The at least one compound of Formula (I) has a bioenhancing effect on CYP1A2 substances and CYP2D6 substances.

In another embodiment, the bioenhancer, the R-capsaicyn inhibitor compound of Formula (I), and the at least one active substance are provided separately. Accordingly, the invention provides a composition comprising
i) at least one compound of Formula (I), and
ii) at least one active substance, tautomers or salts thereof selected from the group of CYP1A2 substances and CYP2D6 substances, wherein the active substance is provided as a separate composition to i). The at least one compound of Formula (I) has a bioenhancing effect on CYP1A2 substances and CYP2D6 substances. The composition is for use as a bioenhancer of the least one active substance selected from CYP1A2 substances and CYP2D6 substances, provided in the separate composition.

The bioenhancing effect on the at least one active substance leads to increased bioavailability of the at least one active substance. This effect is mediated through an inhibition of the CYP enzymes. More specifically, the at least one active substance, tautomers or salts are thereof selected from the group of CYP1A2 substrates (herein called CYP1A2 substances) and CYP2D6 substrates (herein called CYP2D6 substances). In one embodiment, the at least one active substance is a CYP1A2 substance. In another embodiment, the at least one active substance is a CYP2D6 substance. Relevant Cytochrome P450 CYP1A2 substrates (CYP1A2 substances) and Cytochrome P450 CYP2D6 substrates (CYP2D6 substances) for use in accordance with the invention are listed in the online drugbank (www.drugbank.com).

Hence, in one embodiment, the at least one CYP1A2 substance is selected from the group of Bortezomib, Caffeine, Anagrelide, Ropinirole, Theophylline, Lidocaine, Conjugated Estrogens, Ropivacaine, Zolmitriptan, Amitriptyline, Olanzapine, Clozapine, Grepafloxacin, Mirtazapine, Mexiletine, Tacrine, Imipramine, Duloxetine, Flutamide, Haloperidol, Nortriptyline, Fluorouracil, Propranolol, Estrone, Verapamil, Warfarin, Tizanidine, Riluzole, Zileuton, Estradiol, Dacarbazine, Ondansetron, Cyclobenzaprine, Ramelteon, Frovatriptan, Levobupivacaine, Cinacalcet, Pimozide, Propafenone, Clomipramine, Rasagiline, Chlorzoxazone, Chlorpromazine, Mephenytoin, Diclofenac, Azelastine, Acenocoumarol, Aminophenazone, Palonosetron, Melatonin, Imatinib, Quinine, Doxepin, Carbamazepine, Zolpidem, Fluoxetine, Antipyrine, Terbinafine, Cinnarizine, Flunarizine, Naproxen, Phenacetin, Aminophylline, Asenapine, Betaxolol, Estrone sulfate, Guanabenz, Thiothixene, Trifluoperazine, Promazine, Carvedilol, Genistein, Resveratrol, Albendazole, Bromazepam, Dexfenfluramine, Domperidone, Ethanol, Etoposide, Lumiracoxib, Malathion, Maprotiline, Methadone, Methoxyflurane, Mianserin, Pentoxifylline, Perphenazine, Selegiline, Theobromine, Tolperisone, Erlotinib, Cilostazol, Eltrombopag, Clonidine, Muraglitazar, Vadimezan, Nabumetone, Pazopanib, Ulipristal, Lorcaserin, Apixaban, Axitinib, Pomalidomide, Ixazomib, Bendamustine, Rucaparib, Icotinib, Deutetrabenazine, Stiripentol, Voxilaprevir, Enasidenib, Estradiol acetate, Estradiol benzoate, Estradiol cypionate, Estradiol dienanthate, Estradiol valerate, Zotepine, Lofexidine, Tasimelteon, Pirfenidone, Ramosetron, Acetaminophen, Clopidogrel, Fluvoxamine, Tegafur, Dihydralazine, Pimobendan, Ranitidine, Perampanel, (R)-warfarin, 6-O-benzylguanine, 8-chlorotheophylline, Hypoxanthine, Lisofylline, Oxtriphylline, Xanthine, Temafloxacin, Acefylline, Acyclovir, Bamifylline, Bromotheophylline, Bufylline, Cafedrine, Doxofylline, Entecavir, Etamiphylline, Fenethylline, Lobucavir, Peldesine, Pemetrexed, Penciclovir, Pentifylline, Propentofylline, Proxyphylline, Theodrenaline, Propacetamol, Nicotine, Carmustine, Lomefloxacin, Hesperetin, Leflunomide, Rofecoxib, Thiabendazole, Nifedipine, Primaquine, Etoricoxib, Praziquantel, Triamterene, Dasatinib, Capsaicin, Binimetinib, Azathioprine, 8-azaguanine, 9-Deazaguanine, 9-Methylguanine, Disopyramide, Alosetron, Sorafenib, Agomelatine, Tocainide, Triclabendazole, Dapagliflozin, Apremilast, Paroxetine, Tamoxifen, Istradefylline, Lumateperone, Ethinylestradiol, Trimethoprim, Umifenovir, Lopinavir, Selumetinib, Fenfluramine, Abametapir, Pralsetinib, Lonafarnib, Mefenamic acid, Methylene blue, Umbralisib, Febuxostat, Benzocaine, Glycopyrronium, Fexinidazole and Maribavir.

In another embodiment, the at least one CYP2D6 substance is selected from the group of Tramadol, Metoprolol, Venlafaxine, Atomoxetine, Codeine, Timolol, Mexiletine, Duloxetine, Oxycodone, Haloperidol, Dextromethorphan, Tamoxifen, Thioridazine, Paroxetine, Risperidone, Ondansetron, Carvedilol, Doxepin, Desipramine, Flecainide, Clomipramine, Amitriptyline, Imipramine, Propafenone, Aripiprazole, Fluoxetine, Methadone, Tafenoquine, Nortriptyline, Trimipramine, Progesterone, Elagolix, Dosulepin, Acebutolol, Alprenolol, Anisodamine, Arotinolol, Atenolol, Befunolol, Betaxolol, Bevantolol, Bopindolol, Bucindolol, Bufuralol, Bupranolol, Carteolol, Celiprolol, Cloranolol, Epanolol, Esatenolol, Esmolol, Indenolol, Landiolol, Levobetaxolol, Levobunolol, Mepindolol, Metipranolol, Nadolol, Nebivolol, Oxprenolol, Penbutolol, Pindolol, Practolol, Propranolol, Sotalol, Talinolol, Tertatolol, Nifedipine, Vortioxetine, Paliperidone, Dapoxetine, Clozapine, Citalopram, Escitalopram, Fluvoxamine, Levomilnacipran, Nefazodone, Sertraline, Trazodone, Fusidic acid, Galantamine, Ritonavir, Dasabuvir, Buprenorphine, Buspirone, Bepridil, Cinnarizine, Flunarizine, Loperamide, Fluvastatin, Acetaminophen, Propacetamol, Aripiprazole lauroxil, Zuclopenthixol, Fesoterodine, Hydrocodone, Pitolisant, Brexpiprazole, Eliglustat, Amphetamine, Cevimeline, Lidocaine, Mirtazapine, Prochlorperazine, Meperidine, Chlorpromazine, Darifenacin, Clonidine, Nicergoline, Dolasetron, Minaprine, Donepezil, Perphenazine, Phenformin, Almotriptan, Maprotiline, Formoterol, Procainamide, Tolterodine, Promethazine, Perhexiline, Chlorpheniramine, Encainide, Moclobemide, Arformoterol, Ethylmorphine, Tiotropium, Diphenhydramine, Debrisoquine, Gefitinib, Terfenadine, Diltiazem, Chlorzoxazone, Loratadine, Quinine Mephenytoin, Delavirdine, Mesoridazine, Azelastine, Mequitazine, Dexfenfluramine, Oxymorphone, Methotrimeprazine, Dihydrocodeine, Palonosetron, Sertindole, Mianserin, Tesmilifene, Rotigotine, Lisuride, Imatinib, Olanzapine, Bortezomib, Trabectedin, 4-, Methoxyamphetamine, Phenacetin, Sparteine, Ticlopidine, Amoxapine, Chloroquine, Iloperidone, Metamfetamine, Pipotiazine, Tamsulosin, Tetrabenazine, Tipranavir, Dronedarone, Aminophenazone, Eletriptan, Zolpidem, Sildenafil, Amprenavir, Aprindine, Astemizole, Benzatropine, Bupivacaine, Caffeine, Domperidone, Doxazosin, Idarubicin, Methoxyflurane, Nateglinide, Nicotine, Pentamidine, Antipyrine, Piperazine, Quetiapine, Remoxipride, Simvastatin, Tegaserod, Theophylline, Yohimbine, Erlotinib, Cilostazol, Pazopanib, Vilazodone, Repinotan, Benzyl alcohol, Clevidipine, Enclomiphene, Tapentadol, Esmirtazapine, Ixazomib, Rucaparib, Dexchlorpheniramine maleate, Deutetrabenazine, Vernakalant, Valbenazine, Perospirone, Rupatadine, Enasidenib, Letermovir, Opium, Ibrutinib, Asunaprevir, Ipecac, Lofexidine, Lysergic acid diethylamide, Encorafenib, Pimozide, Lorpiprazole, Promazine, Dextropropoxyphene, Epinastine, Midomafetamine, Dacomitinib, Revefenacin, Lisdexamfetamine, Ranolazine, Triclabendazole, Dextroamphetamine, Meclizine, Metoclopramide, Dexchlorpheniramine, Solifenacin, Celecoxib, Butyrfentanyl, Cyclobenzaprine, Selegiline, Midodrine, Istradefylline, Labetalol, Nevirapine, Oxamniquine, Ciclesonide, Bicifadine, Cannabidiol, Azimilide, 5-methoxy-N, N-dimethyltryptamine, Nabiximols, Asenapine, Upadacitinib, Phenytoin, Umifenovir, Lopinavir, Osilodrostat, Remdesivir, Hydroxychloroquine, Ripretinib, Fenfluramine, Oliceridine, Pralsetinib, Pirfenidone, Methylene blue, Benzocaine, Pimavanserin, Glycopyrronium, Belumosudil, Fexinidazole and Ivermectin.

More particularly, the at least one active substance, tautomers or salts thereof may be selected from any of the following CYP1A2 substances in this exemplary, but not exhaustive, non-limiting list: Caffeine, melatonin, diclofenac, duloxetine, tizanidine, acyclovir, haloperidol, carbamazepine, 5-fluorouracil, tamoxifen, etoposide, propranolol, verapamil, rofecoxib, theophylline, ranitidine.

Further, the at least one active substance, tautomers or salts thereof may be selected from any of the following CYP2D6 substances in this exemplary, but not exhaustive, non-limiting list: Caffein, melatonin, duloxetine, phenytoin, tamoxifen, propranolol, atenolol, celiprolol, diltiazem, sparteine, theophylline, loratadine.

The at least one active substance, tautomers or salts thereof may be selected from any of the following substances in this exemplary, but not exhaustive, non-limiting list: Caffeine, melatonin, diclofenac, duloxetine, tizanidine, acyclovir, haloperidol, carbamazepine, 5-fluorouracil, tamoxifen, etoposide, propranolol, verapamil, rofecoxib, theophylline, ranitidine, phenytoin, atenolol, celiprolol, diltiazem, sparteine, loratadine.

Preferably, the at least one active substance, tautomers or salts selected from the group of CYP1A2 substances and CYP2D6 substances, is selected from the group of caffeine, melatonin, diclofenac, duloxetine and tizanedine.

In one embodiment, any one of the following substances are disclaimed from the current invention: diclofenac, acyclovir, haloperidol, carbamazepine, 5-fluorouracil, tamoxifen, etoposide, propranolol, verapamil, rofecoxib, theophylline, ranitidine, phenytoin, atenolol, celiprolol, diltiazem, sparteine, loratadine.

Particularly preferred active substances for use according to the invention may be selected based on their ability to being prepared, rather easily, for co-formulation with the bioenhancer, and on their extensive use and demand.

In one embodiment, the active substance is melatonin. In one embodiment, the active substance is caffeine. In one embodiment, the active substance is diclofenac. In one embodiment, the active substance is duloxetine. In one embodiment, the active substance is tizanedine.

Melatonin is an endogenous hormone produced by the pineal gland that regulates sleep-wake cycles and when provided exogenously has beneficial effects on sleep-onset latency. Melatonin is also implicated in the regulation of mood, learning and memory, immune activity, dreaming, fertility and reproduction. Melatonin is also an effective antioxidant. It is available as an over-the-counter supplement.

Caffeine is a stimulant present in tea, coffee, cola beverages, analgesic drugs, and agents used to increase alertness. It is also used in to prevent and treat pulmonary complications of premature birth. Caffeine is a drug of the methylxanthine class used for a variety of purposes, including certain respiratory conditions of the premature newborn, pain relief, and to combat drowsiness. Caffeine is similar in chemical structure to Theophylline and Theobromine. It can be sourced from coffee beans, but also occurs naturally in various teas and cacao beans, which are different than coffee beans. Caffeine is also used in a variety of cosmetic products and can be administered topically, orally, by inhalation, or by injection.

Diclofenac is an NSAID used to treat the signs and symptoms of osteoarthritis and rheumatoid arthritis. Diclofenac is a phenylacetic acid derivative and non-steroidal anti-inflammatory drug (NSAID). NSAIDs inhibit cyclooxygenase (COX)-1 and -2 which are the enzyme responsible for producing prostaglandins (PGs). PGs contribute to inflammation and pain signalling. Diclofenac, like other NSAIDs, is often used as first line therapy for acute and chronic pain and inflammation from a variety of causes. Diclofenac (generic name) is well-known marketed under brand name Voltaren™.

Duloxetine is a serotonin norepinephrine reuptake inhibitor used to treat generalized anxiety disorder, neuropathic pain, osteoarthritis, and stress incontinence. Duloxetine continues to be investigated for the treatment of pain in cancer, surgery, and more.

Tizanidine is an alpha-2 adrenergic agonist used for the short-term treatment of muscle spasticity. It is a fast-acting drug used for the management of muscle spasm, which may result from the effects of multiple sclerosis, stroke, an acquired brain injury, or a spinal cord injury.

Theophylline is a xanthine used to manage the symptoms of asthma, chronic obstructive pulmonary disease (COPD), and other lung conditions caused by reversible airflow obstruction.

Theobromine is used as a vasodilator, a diuretic, and heart stimulant. And similar to caffeine, it may be useful in management of fatigue and orthostatic hypotension.

In order to further evaluate oral absorption and first-pass effect in humans, an in vitro Caco-2 hepatocyte hybrid system will be used in a study, so as to predict changes in Cmax serum levels of a group of relevant CYP1A2 substances and CYP2D6 substances. This will be performed with and without a single dose of a co-administered CYP1A2 and CYP2D6 inhibitory compound of Formula (I).

The skilled person is familiar with how to produce and manufacture suitable compositions in accordance with good manufacturing practice (GMP). The skilled person is able to handle standard processes for producing stable Formulations and compositions.

The composition of the invention is selected from the group of pharmaceutical compositions, nutraceutical compositions and supplemental compositions. The compound of Formula (I) is administered with the at least one active substance. Hence, the compound of Formula (I) or the composition comprising this, is for use concomitantly or in conjunction with the at least one active substance. Hence, the compound(s) and substance(s) are co-administered or form part of a mixture. More particularly, the compound(s) and the active substance(s) may be part of the same pharmaceutical, nutraceutical or supplement composition. Alternatively, they may be part of two different pharmaceutical, nutraceutical or supplements, respectively. The compound(s) of Formula (I) and the at least one active substance, forming such pharmaceutical, nutraceutical or supplements, may be co-administered in a coordinated fashion. The co-administrations can be simultaneous, sequential, overlapping, in intervals, continuous, or a combination thereof. The compound(s) and the active substance(s) may have the same route of administration or different routes of administration as further detailed below. The compound(s) and active substance(s) may be comprised as different components in a kit as pharmaceuticals, nutraceuticals or supplements.

The at least one active substance may be provided as an active pharmaceutical ingredient (API). Such may be formulated for separate administration to the composition comprising the bioenhancer compound of Formula (I) or may be formulated as part of the same formulation. The API may be provided in the form of an approved medicament. In this regard, a separate composition of the at least one active substance may represent a more flexible and pragmatic solution, as the bioenhancer composition comprising the compound of Formula (I) in principle can be used in combination with any existing and approved therapeutic agent for treatment of any localized pathology. Alternatively, a pharmaceutical composition wherein both the bioenhancer compound of Formula (I) and the at least one active substance (the API) are present, such as formulated into one mixture, would require a more complex development of a novel drug product.

The at least one active substance may alternatively be provided as a nutraceutical or supplemental compound. The development and regulatory process for nutraceutical or supplemental compositions may be easier than for pharmaceuticals. In one embodiment, for such nutraceutical compositions and supplemental compositions of the invention, the compound(s) of Formula (I) and the at least one active substance, tautomers or salts thereof, are formulated as part of the same composition. In another embodiment, for such nutraceutical or supplemental compositions of the invention, the compound(s) of Formula (I) and the at least one active substance, tautomers or salts thereof, are formulated as separate compositions. In one example, the active substance is melatonin, and this is provided as a medicament, or preferably as a nutraceutical or supplemental compound. The compound of Formula (I) and melatonin are either formulated as part of the same composition or are formulated as separate compositions. In one embodiment, such formulation comprising a compound of Formula (I) and melatonin may be for conditions, disorders or diseases selected from the group of sleeping issues, such as sleep-onset latency, and may be used to easier get to sleep, and improve the sleep. In another example, the active substance is caffein, and this is provided as a medicament, or preferably a nutraceutical or supplemental compound. The compound of Formula (I) and caffein are either formulated as part of the same composition or are formulated as separate compositions. In one embodiment, such formulation comprising a compound of Formula (I) and caffein may be for use as an energy supplement to restore mental alertness or wakefulness.

Said supplement comprises, but is not limited to, at least one of: dietary supplement, nutritional supplement, nutraceutical supplement, over-the-counter supplement and/or pharmaceutical grade supplement.

The composition of the invention may comprise at least one other active substance, different from a CYP1A2 substance and/or CYP2D6 substance. Such at least one other active substance may be selected from essential nutrients, such as any of the following groups: vitamins, fatty acids, proteins, peptides, amino acids, carbohydrates, minerals, trace elements and/or colouring agents. Said vitamins include, but are not limited to at least one of vitamin A, B1, B2, B3, B5, B6, B7, B9, B12, C, D, E and/or K. Said amino acids include, but are not limited to at least one of alanine, arginine, asparagine, aspartic acid, cysteine, glutamine, glutamic acid, glycine, histidine, isoleucine, lysine, methionine, phenylalanine, proline, serine, threonine, tryptophan, tyrosine and/or valine. Said minerals include, but are not limited to at least one of potassium, chlorine, sodium, calcium, phosphorous, magnesium, iron, zinc, manganese, copper, iodine, chromium, molybdenum, selenium and/or cobalt.

The skilled person is familiar with the process of pharmaceutical, nutraceutical and supplement manufacture in accordance with GMP. The skilled person is able to either produce formulations comprising the compound of Formula (I) with the active substance or produce compositions wherein the compound of Formula (I) and the active substance are in separate pharmaceutical, nutraceutical or supplement compositions. The skillset of said skilled person allows said person to determine, without undue burden, which option is more suitable in each variant of pharmaceutical, nutraceutical or supplement.

Use of the compounds of Formula (I) together with the at least one active substance, tautomers or salts thereof, results in improved bioavailability of the active substance(s). The applicant has found that capsaicyns of Formula (I) exert inhibitory effects on Cytochrome P450 CYP1A2 and CYP2D6 enzymes, and hence that use of CYP1A2 or CYP2D6 substances with such capsaicyns result in a bioenhancer effect for those substances. An effect is increased uptake of said active substance. The half-life of the at least one active substance is improved when taken in conjunction with a compound of Formula (I), compared to when taken without such capsaicyn. The effect is particularly important for active substances taken orally, to directly inhibit the metabolism and degradation in the liver by the relevant enzymes. Accordingly, the use of a compound of Formula (I) may lower the amount of active substance needed to achieve similar results as the higher amount of active substance without any compound of Formula (I). Accordingly, the dose of the at least one active substance may be reduced.

Hence, an advantage of said composition for use in treatment or prevention of a condition, disorder or disease is a reduction of the required amount of active substance for achieving the desired outcome compared to the required amount in the absence of said composition. A reduced amount of substance reduces the risk of developing side effects and/or reduces the severity of side effects. The dosage may also be smaller compared to standard dosages, resulting in fewer administrations, longer between administrations or smaller amounts to ingest for each administration. In one embodiment, it is expected that the dosage of active substance can be reduced with at least 5%, such as at least 10%, compared to when administered without the compound of Formula (I), to achieve the same therapeutic effect. These factors increase the subject compliance, therefore making it more likely for a subject to, for instance, complete a recommended treatment regime.

As a result of increased bioavailability and potentially a reduced dosage, when administering the active substance with the compound of Formula (I), reduced side effects of the active substance may be achieved. As an example, diclofenac (Voltaren) is known to cause some unwanted effects, most of them not requiring medical intention. However, one potentially severe side effect seen typically for athletes using high doses of the medicament, is heart disorders, e.g. pain in the chest, heartburn, and even heart attack. The compound of Formula (I) used with a CYP1A2 or CYP2D6 substance, such as diclofenac, can reduce the side effects of the substance. In one embodiment, the compound of formula (I), such as e.g. phenylcapsaicyn or a composition comprising such, is administered orally, and co-administering a CYP1A2 or CYP2D6 substance, such as diclofenac, in the form of a separate composition. Diclofenac may e.g. be administered orally or as a topical medicament.

The use of said composition may also reduce drug resistance. A known problem is resistance to drugs. Bioenhancers lowers the amount of active substance needed, thus reducing the overall use of the active substance, which is an important part of preventing resistance. Bioenhancers may also increase the uptake or reduce the extrusion of said active substance, resulting thus in a more efficient treatment regime.

The reduced need of active substance may result in a reduced demand of raw materials for said active substance. This results in positive ecological implications as the materials may be important for their environment. Reduced demand of scare materials is also advantageous. The supply will also be less affected by fluctuations in raw material supply, which is subject to slow and rapid changes in availability. Overall, the reduced amount of active substance can lower the cost of treatments, thus minimising the barrier between those who are financially strong and can afford treatments and those who are financially challenged and may normally not be able to afford treatment. This may reduce their disease burden and/or mortality and is beneficial for societal health.

Furthermore, a reduction of ingested active substance leads to reduced excretion of said active substance and metabolites thereof. If said substance and/or metabolites thereof cause environmental pollution or exert other harmful actions in the environment, a reduced excretion leads to less pollution and/or other harmful actions.

As a further effect of the invention, the at least one active substance is less aggressive, and more efficient, than when used without the compound of Formula (I).

Further, the cost of the total amount of active substance is subsequently reduced as there is a reduced demand of ingredients, expensive ingredients, and/or ingredients of limited reserves. This may in turn reduce the overall price of said pharmaceutical, nutraceutical or supplement.

The compound(s) and the active substance(s) may have the same route of administration or different routes of administration, if present in separate compositions. In the embodiments and/or aspects herein, the routes of administration may be independently selected from, but not limited to: parenteral, which comprises intravenous, intramuscular, subcutaneous and intradermal; inhalational; dermal/topical; oral; sublingual; nasal, intraocular; enteral; rectal; and/or intrathecal. Preferably, the route of administration(s) is oral, sublingual, enteral and/or rectal. More preferably, the route of administration is oral, preferably for the compound(s) and the active substance(s). An advantage of administrations such as oral administration is the low level of invasiveness, causing less stress in the subject than more invasive administration routes, such as parenteral. Further, the effect is particularly important for active substances taken orally, to directly inhibit the metabolism and degradation in the liver by the relevant enzymes.

In some embodiments, the compound of Formula (I) or composition comprising this, is administered orally. In some embodiments, this is administered with a meal or before a meal. In some embodiments, also the at least one active substance is administered orally.

The therapeutically effective dose of the composition according to the invention can be administered in a single dose or in divided doses, such as once, twice or more times a day, once every two days, once every three days, twice a week or once a week, or as deemed appropriate by a medical professional. In certain embodiments, the composition according to the invention is administered once daily. In other embodiments, the composition according to the invention is administered twice daily. In some embodiments the dosage and the frequency of administration with the composition according to the invention is determined by a medical professional, based on factors including, but not limited to, the stage of the disorder, condition or disease, the severity of symptoms, the route of administration, the age, body weight, general health, gender and/or diet of the subject, and/or the response of the subject to the treatment. In some embodiments, the therapeutically effective dose is administered at regular intervals. In other embodiments, the dose is administered when needed or sporadically. The composition according to the invention may be administered by a medical professional. The composition according to the invention may, depending on factors such as formulation and route of administration, be administered with food or without food. In some embodiments, the composition according to the invention is administered at specific times of day.

Preferred unit dosage formulations are those containing a therapeutically effective dose, as hereinbefore recited, or an appropriate fraction thereof, of a compound or composition according to the invention. A composition of the invention may be presented in unit dosage form as a single dose wherein all active and inactive ingredients, i.e. compound of Formula (I) and an active CYP1A2 or CYP2D6 substance, are combined in a suitable system and components do not need to be mixed before administration. Alternatively, a composition may be presented as a kit such as disclosed above, and may contain instructions for storing, preparing, administering and/or using the composition. Furthermore, the two may be provided completely separate; the bioenhancer of Formula (I) is provided, and is to be taken with an existing, separately provided, active substance selected from CYP1A2 or CYP2D6 substances.

Another aspect of the invention is the use of at least one compound of Formula (I). As disclosed above, this is directed to the use of at least one compound of Formula (I) as a bioenhancer of a at least one active substance selected from the group of CYP1A2 and CYP2D6 substances. The bioenhancing effect leads to an increased bioavailability of the at least one active substance.

Another aspect is a composition for use in treatment or prevention of a condition, disorder or a disease comprising a compound of Formula (I), wherein the compound of Formula (I) act as a bioenhancer of at least one active substance selected from CYP1A2 substances and CYP2D6 substances used in the treatment or prevention.

It may be appreciated that the use of a compound of Formula (I) as a bioenhancer not necessarily is restricted by particular conditions, disorders or diseases. The compound does not treat the condition, disorder or disease itself, but the bioenhancing properties arise from interactions increasing the bioavailability of the active substance, particularly by its inhibiting properties on the CYP1A2 and CYP2D6 enzymes. Such bioenhancing properties may therefore be dependent on the molecular structure of the active substance and its physiological interactions with a target subject, not on the nature of the condition, disorder or disease.

Generally, however, such condition, disorder or a disease may be, but are not limited to, mental alertness during fatigue or drowsiness, sleeping issues, infections, inflammations, anxiety, pain, and/or depression.

In one embodiment, the active substance is melatonin, and the condition, disorder or disease is selected from the group of sleeping issues, such as sleep-onset latency, and may be used to easier get to sleep, and improve the sleep.

In one embodiment, the active substance is caffeine, and the condition, disorder or disease is selected from the group of fatigue, drowsiness, headache, migraine, and low energy-feeling. The substance may be used as an energy supplement to restore mental alertness or wakefulness.

In one embodiment, the active substance is diclofenac, and the condition, disorder or disease is selected from the group of inflammations, e.g. inflammatory arthritis, and pain.

In one embodiment, the active substance is duloxetine, and the condition, disorder or disease is selected from the group of depression and anxiety, nerve pain such as fibromyalgia, bone or muscle pain.

In one embodiment, the active substance is tizanedine, and the condition, disorder or disease is selected from the group of muscle spasticity and pain sensations. Tizanedine act as a muscle relaxant, blocking nerve impulses.

The compositions may also further comprise bioenhancers other than the compound of Formula (I). Such additional bioenhancer may be one or more of, but is not limited to, the following group: curcumin, piperine, quercetin, gingerols, allicin, glycyrrhizin, genistein, sinomenine, Stevia rebaudiana, Aloe vera, lysergol, Carum carvi, niaziridin, capsaicin, naringin, Zingiber officinale, Ammannia multiflora, capmul, and/or cow urine distillate.

Another aspect is a method of treating or preventing a condition, disorder or a disease in a subject. The method comprises administering an effective amount of
i) at least one compound of Formula (I)
wherein R is alkyl, trifluoromethyl, cycloalkyl, phenyl, or halogen, when said substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl, or a composition comprising such compound;
wherein the compound of Formula (I) act as a bioenhancer of at least one active substance selected from CYP1A2 substances and CYP2D6 substances used in the treatment or prevention.

Furthermore, the invention provides a method for increasing the bioavailability of an active substance in a subject, wherein the active substance is selected from CYP1A2 substances and CYP2D6 substances, the method comprises a step of administering a compound of Formula (I) to the subject, the compound acting as a bioenhancer of the at least one active substance. As disclosed, the active substance and the compound of Formula (I) may be formulated as one composition, or as separate compositions, and may hence be administered separately or combined.

By subject we mean humans, and this may comprise at least one of different groups such as: male, female, infants, children, teenagers, adults, elderly, humans with pre-existing conditions, humans without pre-existing conditions, and/or humans pre-disposed for conditions.

Another aspect is a compound of Formula (I) in an amount sufficient for achieving a bioenhancing effect on at least one active substance, tautomers or salts thereof.

The compound of Formula (I) may be included in the compositions, in concentrations providing the disclosed bioenhancing effect. The concentration of the compound of Formula (I), as provided by parts per million (ppm), is such as, but not limited to: 1-500 ppm, 5-250 ppm, 10-100 ppm, 10-75 ppm, 10-50 ppm, 5-50 ppm, 1-50 ppm. It is routine work to select suitable amounts to be incorporated into said compositions. A skilled person is able to do so without undue burden.

The compositions disclosed herein may comprise at least one excipient. Excipients are pharmaceutically inactive ingredients applied to compositions to ensure that said compositions may be safe, convenient and/or acceptable for use. Such excipients include, but are not limited to: anti-adherents, binders, coatings, colour agents, disintegrants, flavouring agents, glidants, lubricants, preservatives, sorbents, sweeteners, pH modifiers, fillers, antioxidants, viscosity modifiers, absorbents, diluents or vehicles. It is routine work to select suitable excipients including selecting suitable amounts and incorporate said excipients into said compositions. A skilled person is able to do so without undue burden. apply to the other aspects of the invention.

The embodiments and features described in the context of one aspect, e.g. for the aspect directed to the compound of Formula (I) or the composition comprising such, also apply to the other aspects of the invention, such as in the use thereof or in a method of treatment. The varieties of compounds and active substance options, and combinations, disclosed for one aspect hence also apply to the other aspects.

The invention shall not be limited to the shown embodiments and examples. While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is to be understood that various alternatives to the embodiments described herein can be employed in practicing the disclosure.

It is to be understood that every embodiment of the disclosure can optionally be combined with any one or more of the other embodiments described herein.

It is to be understood that each component, compound, particle, or parameter disclosed herein is to be interpreted as being disclosed for use alone or in combination with one or more of each and every other component, compound, or parameter disclosed herein. It is further to be understood that each amount/value or range of amounts/values for each component, compound, or parameter disclosed herein is to be interpreted as also being disclosed in combination with each amount/value or range of amounts/values disclosed for any other component(s), compound(s), or parameter(s) disclosed herein, and that any combination of amounts/values or ranges of amounts/values for two or more component(s), compound(s), or parameter(s) disclosed herein are thus also disclosed in combination with each other for the purposes of this description. Any and all features described herein, and combinations of such features, are included within the scope of the present invention provided that the features are not mutually inconsistent.

### EXAMPLES

### Example 1: Evaluation of the Direct Inhibitory Potential of Phenylcapsaicyn Against Human Cytochrome P450 Isoforms Using Human Liver Microsomes

### Introduction

The objective of the study was to evaluate the direct and time-dependent inhibitory potential of phenylcapsaicyn towards seven human cytochrome P450 isoforms (CYP1A2, CYP2B6, CYP2C8, CYP2C9, CYP2C19, CYP2D6, and CYP3A4/5) using pooled human liver microsomes and CYP-specific marker substrate reactions. The substrate reactions were CYP1A2-mediated phenacetin O-deethylation, CYP2B6-mediated bupropion hydroxylation, CYP2C8-mediated amodiaquine N-deethylation, CYP2C9-mediated diclofenac 4′-hydroxylation, CYP2C19-mediated S-mephenytoin 4′-hydroxylation, CYP2D6-mediated dextromethorphan O-demethylation and CYP3A4/5-mediated midazolam 1′-hydroxylation and testosterone 6β-hydroxylation.

### Abbreviations

CAN Acetonitrile
% CV Coefficient of Variation
CYP Cytochrome P450
CYP1A2 Cytochrome P450 Isoform 1A2
CYP2B6 Cytochrome P450 Isoform 2B6
CYP2C8 Cytochrome P450 Isoform 2C8
CYP2C9 Cytochrome P450 Isoform 2C9
CYP2C19 Cytochrome P450 Isoform 2C19
CYP2D6 Cytochrome P450 Isoform 2D6
CYP3A4/5 Cytochrome P450 Isoform 3A4/5
DMSO Dimethyl sulfoxide
ESI Electrospray Ionization
HLM Human liver microsomes
IC50 Inhibitor concentration which inhibits maximal activity by 50%
IS Internal Standard
KPi 100 mM potassium phosphate buffer, pH=7.4
LC-MS/MS Liquid chromatography-mass
MeOH Methanol
MRM Multiple Reaction Monitoring
NA Not Applicable
NI PAR No Inhibition Peak area ratio
rpm Revolutions per minute
SD Standard Deviation
Thio TEPA N,N′,N″-Triethylenethiophosphoramide
VC Vehicle Control (0 μM test article)
% VC Percent of Vehicle Control

### Materials and Methods

A stock solution of 50 mM phenylcapsaicyn in DMSO was prepared and stored at −20° C. Pooled human liver microsomes (HLM) (Lot #: 1410230) were used from XenoTech, LLC (USA). Buffers were prepared with reagents purchased from Sigma Chemicals (USA). 96-well polypropylene assay plates and rubber sealing mats were purchased from Fisher Scientific (USA).

The following CYP Probe substrates were used:

| | | | | MW/FW |
|---|---|---|---|---|
| Substance | Supplier | Cat# | Purity (%) | (g/mol) |
| (+)-Benzylnirvanol | TRC | B285775 | 98.0 | 294.35 |
| Furafylline | TRC | F864070 | 96.0 | 260.25 |
| Itraconazole | Sigma | I6657 | 100.0 | 705.63 |
| Montelukast | Sigma | SML0101 | 99.0 | 635.17 |
| Quinidine | Sigma | 22600 | 100.0 | 324.42 |
| Sulfaphenazole | Sigma | S0758 | 99.5 | 314.4 |
| Tranylcypromine | TRC | P319680 | 98.0 | 169.65 |

Preparation of reagents: Potassium phosphate buffer (100 mM, pH 7.4) was prepared by first diluting 1 M solutions of monobasic potassium phosphate and dibasic potassium phosphate with ultrapure water to 0.1 M. The 100 mM solutions were combined in a ratio (~3:1) dibasic:monobasic potassium phosphate such that the final pH of the buffer was 7.4. The resulting 100 mM potassium phosphate buffer, pH=7.4 (KPi) was sterile-filtered and stored refrigerated (4° C.).

HLM (XenoTech, LLC) at 20 mg/mL was diluted in the reaction mix to 0.1 mg/mL for incubation with probe substrates.

Preparation of stock solutions: A single weigh out was used for all solution preparations. Molecular weights of all compounds were corrected for salt forms, water or solvent content, and purity for stock solution preparation. The final concentration of DMSO in all reactions was ≤0.1% and for ACN or MeOH the final concentration was ≤1%.

A stock solution of 50 mM phenylcapsaicyn (Lot No. P245-141-2) in DMSO was prepared and aliquoted into polypropylene microfuge tubes and stored at −20° C. Each vial was thawed once on the day of the experiment and discarded. All solutions containing phenylcapsaicyn were stored in amber vials or microfuge tubes and protected from light. Working solutions of phenylcapsaicyn were prepared at 200 μM (4× the highest incubation concentration), by adding 2.4 μL of the DMSO stock solution into 100 mM potassium phosphate buffer, pH=7.4 (KPi) to give a final volume of 600 μL. From this working solution, 1:10 serial dilutions were made in KPi containing organic solvent to generate the remaining working solutions of phenylcapsaicyn (4× of final incubation concentration) for incubation. All dilutions were made such that the final concentration of organic solvent in the reactions was 0.1% DMSO and ≤0.9% ACN or ≤0.9% of a mixture of ACN and MeOH (for CYP2C8). The concentrations of organic solvent were consistent through all the samples for any given set of CYP isoform reactions. Six final concentrations of phenylcapsaicyn were evaluated in triplicate, 0, 0.005, 0.05, 0.5, 5.0, and 50 μM phenylcapsaicyn, using all eight CYP probe substrates.

Stock solutions (Stock A) of positive control direct inhibitors were made in DMSO for all except montelukast which was made in MeOH. Concentrations were as follows: furafylline (CYP1A2) and tranylcypromine (CYP2B6) were 100 mM, sulfaphenazole (CYP2C9), (+)-benzylnirvanol (CYP2C19) and itraconazole (CYP3A4/5 with midazolam) were 25 mM, montelukast (CYP2C8) and quinidine (CYP2D6) were 10 mM, and itraconazole (CYP3A4/5 with testosterone) was 5 mM. Working solutions (Stock B) were prepared at 4× the final incubation concentration in KPi containing organic solvent. From the Stock B solution, 1:10 serial dilutions were made to generate 5 concentrations of positive controls (4× the final incubation concentration). These stock solutions were added directly to the reaction mix to yield the final substrate concentrations shown in Table 1.

TABLE 1
Final concentrations of CYP substrates and positive controls
by CYP isoform and metabolite for direct inhibition

| | | | | | Final |
|---|---|---|---|---|---|
| | | Final | | | Highest |
| | | Concentration | | | Concentration |
| CYP | | of Substrate | | Positive | of Positive |
| isoform | Substrate | (μM) | Metabolite Analyzed | Control | Control (μM) |
| 1A2 | Phenacetin | 60 | Acetaminophen | Furafylline | 100 |
| 2B6 | Bupropion | 80 | Hydroxybupropion | Tranylcypromine | 100 |
| 2C8 | Amodiaquine | 2 | N-Desethylamodiaquine | Montelukast | 10 |
| 2C9 | Diclofenac | 5 | 4′-Hydroxydiclofenac | Sulfaphenazole | 25 |
| 2C19 | S-Mephenytoin | 80 | 4′-Hydroxymephenytoin | (+)-Benzylnirvanol | 25 |
| 2D6 | Dextromethorphan | 5 | Dextrorphan | Quinidine | 20 |
| 3A4/5 | Midazolam | 2 | 1′-Hydroxymidazolam | Itraconazole | 25 |
| 3A4/5 | Testosterone | 50 | 6β-Hydroxytestosterone | Itraconazole | 5 |

### CYP Inhibition Incubations for Direct Inhibition:

Reactions were assembled in conical bottom, polypropylene 96-well plates. A reaction mix containing KPi, HLM, any organic solvent needed, and the substrate stock solution was mixed and aliquoted (104 μL) into the designated wells of the 96-well plate. Next, 40 UL of 4× inhibitor dilution or blank was added to the appropriate wells and mixed. The plate was placed on a rotary shaker at 150 rpm in a 37° C. incubator to equilibrate for 5 minutes. Reactions were started by the addition of 16 μL of 10 mM NADPH (10×) and returned to the incubated shaker. The total incubation volume was 160 μL. Reactions incubated for 5 minutes (midazolam), 10 minutes (phenacetin, amodiaquine, dextromethorphan, and testosterone), 15 minutes (bupropion and diclofenac), or 25 minutes (S-mephenytoin). phenylcapsaicyn incubations were conducted in triplicate and positive control incubations were conducted in duplicate. At the end of incubation, reactions were terminated by taking 100 UL of each reaction and adding to a 96-well plate containing an equal volume of ice cold ACN with a cocktail of stable labeled internal standards. The 96-well plate containing the terminated reactions was sealed with a rubber mat and vortexed on high for 4 minutes then centrifuged at 3000 rpm at 4° C. for 10 minutes. Phenacetin and midazolam were pooled by taking 100 μL aliquots of corresponding wells and adding to a fresh deep-well 96-well block. Bupropion and diclofenac reactions were pooled analogously to the phenacetin and midazolam reactions. All other reactions were analyzed discreetly by taking 150 μL from each well and adding to the corresponding well of a fresh deep-well 96-well block. The plates were sealed with rubber mats, vortexed on medium setting for 2 minutes, and centrifuged as above for 5 minutes. The samples were analyzed for determination of relative metabolite levels using the peak area ratio (PAR) of the metabolite peak to that of the internal standard peak.

Data analysis: The relative amount of each metabolite was calculated by determining the PAR of the metabolite peak to that of the corresponding internal standard. The degree of inhibition was calculated by comparing the formation of metabolites in the presence of different concentrations of phenylcapsaicyn (positive inhibitor) versus vehicle control. IC50 values were calculated using the 4 parameter dose response equation in GraphPad Prism Version 6.04.

### Results and Discussion

The direct inhibitory activity was measured using six concentrations of phenylcapsaicyn in triplicate, 0, 0.005, 0.05, 0.5, 5.0, and 50 μM.

Tables 2-6 below provide the CYP activity for the different isoforms. CYP1A2 was strongly inhibited by phenylcapsaicyn relative to the positive control direct inhibitor, furafylline (Tables 2 and 6). The IC50 for furafylline inhibition of CYP1A2 was 5.5 μM and the IC50 for phenylcapsaicyn inhibition of CYP1A2 was 0.67 μM.

CYP2D6 was moderately inhibited by phenylcapsaicyn relative to the positive control direct inhibitor, quinidine (Tables 2 and 4). The IC50 for quinidine inhibition of CYP2D6 was 0.09 μM and the IC50 for phenylcapsaicyn inhibition of CY2D6 was 1.1 μM.

Phenylcapsaicyn caused no significant inhibition of CYP2B6, CYP2C8, CYP2C9, CYP2C1, and CYP3A4/5 with IC50 values of >50 μM in all cases. The positive control direct inhibitors were fit to the 4 parameter dose response model.

The IC50 values for furafylline (CYP1A2), tranylcypromine (CYP2B6), montelukast (CYP2C8), sulfaphenazole (CYP2C9), (+)-benzylnirvanol (CYP2C19), quinidine (CYP2D6), and itraconazole (CYP3A4/5) were 5.5, 3.4, 0.2, 0.21, 0.33, 0.09, 0.21 (midazolam as substrate), and 0.03 μM (testosterone as substrate), respectively. These values are within the range of literature values commonly accepted in the industry [1-5].

Phenylcapsaicyn was found to be a strong inhibitor of CYP1A2 and moderate inhibitor of CYP2D6 relative to the positive control. Some approximations and assumptions can be made to help put the data in context, but the validity of the outcomes would need further validation. Using the Cheng-Prusoff relationship between IC50 and Ki [6] where IC50=2×Ki, and assuming competitive inhibition, an estimate of the Ki can be made. This relationship yields a Ki value of 0.34 μM for CYP1A2 and 0.55 μM for CYP2D6. A rough approximation can also be used to relate the Ki to the expected Cmax to determine the relative risk for in vivo drug interactions possible with the test article. The likelihood of an in vivo interaction is projected based on the [I]/Ki ratio where [I] is the mean steady-state Cmax value for total drug (bound plus unbound) following administration of the highest proposed clinical dose. An [I]/Ki ratio >1 is likely to yield a drug interaction in vivo, a possible interaction occurs when 1>[I]/Ki>0.1, and when 0.1>[I]/Ki, a drug interaction is unlikely.

Even at the ultra-low minimum dose of 5 mg resulting in a Cmax of ~0.6 μM, this gives a [I]/Ki of 18 for CYP1A2 and 1.1 for CYP2D6. These values are >1, indicating that an in vivo drug bio enhancing effect with concomitantly administered drugs metabolized by CYP1A2 and CYP2D6 is likely.

TABLE 2
CYP Isoform activities in incubations treated with varying phenylcapscin
concentrations (0 to 50 μM). CYP activity expressed as % Vehicle Control

| Phenylcapsaicyn | | | | | | | | |
|---|---|---|---|---|---|---|---|---|
| Concentration | CYP | CYP | CYP | CYP | CYP | CYP | CYP | CYP |
| (μM) | 1A2 | 2B6 | 2C8 | 2C9 | 2C19 | 2D6 | 3A4/5^{a} | 3A4/5 ^{b} |
| 0 (VC) | 100.0 | 100.0 | 100.0 | 100.0 | 100.0 | 100.0 | 100.0 | 100.0 |
| 0.005 | 99.9 | 92.1 | 102.8 | 101.0 | 99.3 | 99.2 | 96.5 | 95.6 |
| 0.05 | 94.2 | 95.6 | 100.7 | 102.9 | 97.7 | 97.6 | 97.1 | 100.4 |
| 0.5 | 60.9 | 94.2 | 100.9 | 99.0 | 103.7 | 72.7 | 98.2 | 100.5 |
| 5.0 | 16.4 | 86.7 | 102.6 | 99.9 | 99.4 | 29.9 | 104.1 | 101.3 |
| 50 | 8.0 | 75.3 | 97.8 | 94.7 | 84.8 | 14.8 | 97.8 | 93.8 |
| IC50 (μM) | 0.67 | >50 | >50 | >50 | >50 | 1.12 | >50 | >50 |

^{a}Midazolam was used as the probe substrate.
^{b} Testosterone was used as the probe substrate.
VC—Vehicle Control

TABLE 3
CYP3A4/5^{a} Activity in Incubations treated with
direct inhibitor positive control (0 to 5 μM).
CYP Activity expressed as % Vehicle Control.

| Positive Control | |
|---|---|
| Concentration (μM) | CYP3A4/5 |
| 0 (VC) | 100.0 |
| 0.0005 | 93.1 |
| 0.005 | 79.2 |
| 0.05 | 40.1 |
| 0.5 | 11.4 |
| 5.0 | 4.3 |
| IC50 (μM) | 0.03 |
| Inhibitor | Itraconazole |

^{a}Testosterone was used as the probe substrate.

TABLE 4
CYP2C8 and CYP2D6 Activity in Incubations treated
with direct inhibitor positive control (0 to 5 μM).
CYP Activity expressed as % Vehicle Control.

| Positive Control | | |
|---|---|---|
| Concentration (μM) | CYP2C8 | CYP2D6 |
| 0 (VC) | 100.0 | 100.0 |
| 0.001 | 96.0 | 95.9 |
| 0.01 | 91.3 | 85.2 |
| 0.1 | 66.2 | 48.3 |
| 1.0 | 13.3 | 12.0 |
| 10 | 1.3 | 4.5 |
| IC50 (μM) | 0.20 | 0.09 |
| Inhibitor | Montelukast | Quinidine |

TABLE 5
CYP2C9 and CYP2C19 Activity in Incubations treated
with direct inhibitor positive controls (0 to 25
μM). CYP Activity expressed as % Vehicle Control.

| Positive Control | | |
|---|---|---|
| Concentration (μM) | CYP2C9 | CYP2C19 |
| 0 (VC) | 100.0 | 100.0 |
| 0.0025 | 102.1 | 103.3 |
| 0.025 | 95.7 | 90.5 |
| 0.25 | 47.2 | 58.2 |
| 2.5 | 10.1 | 13.2 |
| 25 | 1.9 | 2.6 |
| IC50 (μM) | 0.21 | 0.33 |
| Inhibitor | Sulfaphenazole | Benzylnirvanol |

TABLE 6
CYP1A2 and CYP2B6 Activity in Incubations treated
with direct inhibitor positive control (0 to 100
μM). CYP Activity expressed as % Vehicle Control.

| Positive Control | | |
|---|---|---|
| Concentration (μM) | CYP1A2 | CYP2B6 |
| 0 (VC) | 100.0 | 100.0 |
| 0.01 | 103.9 | 92.3 |
| 0.1 | 110.2 | 98.3 |
| 1 | 88.5 | 76.9 |
| 10 | 41.0 | 24.7 |
| 100 | 10.1 | 4.2 |
| IC50 (μM) | 5.5 | 3.4 |
| Inhibitor | Furafylline | Tranylcypromine |

### Conclusion

As shown in this study phenylcapsaicyn is a direct and time-dependent inhibitor of CYP1A2 and CYP2D6 at concentrations up to 50 μM under conditions utilized in this study.

At projected use doses and based on [I]/Ki calculated values >1, a drug bio enhancing effect with concomitantly administered substances or drugs that are metabolized by CYP1A2 and CYP2D6 is likely.

### REFERENCES

1. Obach R S, Walsky R L, Venkatakrishnan K, Gaman E A, Houston J B, Tremaine L M The utility of in vitro cytochrome P450 inhibition data in the prediction of drug-drug interactions. J Pharmacol Exp Ther. 2006; 316:336-348.
2. Walsky R L, Obach R S. Verification of the selectivity of (+)N-3-benzylnirvanol as a CYP2C19 inhibitor. Drug Metab Dispos. 2003; 31:343.
3. Walsky R L, Obach R S, Gaman E A, Gleeson J R, Proctor W R. Selective inhibition of human cytochrome P4502C8 by montelukast. Drug Metab Dispos. 2005; 33:413-418.
4. Turpeinen M, Nieminen R, Juntunen T, Taavitsainen P, Raunio H, Pelkonen O. Selective inhibition of CYP2B6-catalyzed bupropion hydroxylation in human liver microsomes in vitro. Drug Metab Dispos. 2004; 32:626-631.
5. Lee K S & Kim S K. Direct and metabolism-dependent cytochrome P450 inhibition assays for evaluating drug-drug interactions. J Appl Toxicol. 2011); 33:100-108.
6. Cheng Y & Prusoff W H. Relationship between the inhibition constant (KI) and the concentration of inhibitor which causes 50 percent inhibition (150) of an enzymatic reaction. Biochem. Pharmacol. 1973; 22:3099-3108.

### Example 2: Planned Study-Oral Absorption and First-Pass Effect in Humans of Phenylcapsaicyn as Bioenhancer for Selected Active Substances

The suggested study will include use of an in vitro Caco-2 hepatocyte hybrid system to estimate oral absorption and first-pass effect in humans, so as to predict changes in Cmax serum levels of certain CYP1A2 substances and CYP2D6 substances with and without a single dose of co-administered CYP1A2 and CYP2D6 inhibitory compound, phenylcapsaicyn.

The following CYP1A2 substances and CYP2D6 substances will form part of the study: caffeine, melatonin, diclofenac, duloxetine and tizanidine.

### Example 3 (Prospective): Alternative Compounds of Formula (I) for Use as Bioenhancers

In order to show that the invention is applicable for a variety of compounds of Formula (I), several compounds may be manufactured and explored for their bioenhancing effect on CYP1A2 substances and CYP2D6 substances.

The following compounds of the invention may be synthesized and tested:
methylcapsaicyn, ethylcapsaicyn, propylcapsaicyn, butylcapsaicyn.

The suggested compounds above may accordingly be studied, e.g. as shown in Example 1 for phenylcapsaicyn, to evaluate the direct and time-dependent inhibitory potential of the compounds towards the human cytochrome P450 isoforms CYP1A2 and CYP2D6.

1-22. (canceled)

23. A method of using a compound of Formula (I) 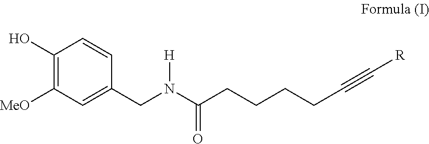
wherein R is alkyl, trifluoromethyl, cycloalkyl, phenyl, or halogen, and when the substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthiol, cyano, cycloalkyl, cycloalkenyl, halo, hydroxy, oxo, nitro, or trifluoromethyl,
as a bioenhancer of at least one active substance selected from the group consisting of Pirfenidone, Ramelteon, Terbinafine, Zolpidem, caffeine, melatonin, duloxetine and tizanidine, or tautomers or salts thereof,
the method comprising orally administering the compound of Formula (I) and the at least one active substance selected from the group consisting of Pirfenidone, Ramelteon, Terbinafine, Zolpidem, caffeine, melatonin, duloxetine and tizanidine, or tautomers or salts thereof, to a subject.

24. The method of using a compound according to claim 23, wherein R is a C1-C6 alkyl or phenyl.

25. The method of using a compound according to claim 23, wherein R is phenyl.

26. The method of using a compound according to claim 23, wherein the at least one active substance is selected from the group consisting of caffeine, melatonin, duloxetine and tizanidine, or tautomers or salts thereof.

27. The method of using a compound according to claim 23, wherein the at least one active substance is melatonin or caffeine.

28. The method of using a compound according to claim 23, for increasing the bioavailability of the at least one active substance.

29. The method of using a compound according to claim 23, wherein the compound of Formula (I) and the at least one active substance are orally co-administered to the subject or form part of a mixture and are orally co-administered to the subject.

30. A composition comprising:
i) at least one compound of Formula (I) 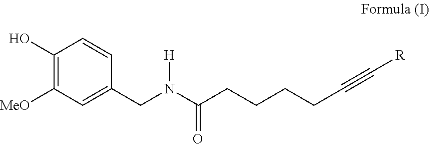
wherein R is alkyl, trifluoromethyl, cycloalkyl, phenyl, or halogen, and when the substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl; and
ii) at least one active substance selected from the group consisting of Pirfenidone, Ramelteon, Terbinafine, Zolpidem, caffeine, melatonin, duloxetine and tizanidine, or tautomers or salts thereof;
wherein the compound of Formula (I) has a bioenhancing effect on the at least one active substance.

31. The composition according to claim 30, wherein the at least one active substance is selected from the group consisting of caffeine, melatonin, duloxetine and tizanidine, or tautomers or salts thereof.

32. The composition according to claim 30, wherein the two components i) and ii) are mixed into one composition formulation.

33. The composition according to claim 30, wherein the composition is a combined composition for simultaneous, separate or sequential use, the composition comprising i) a first composition comprising the compound of Formula (I), and ii) a second composition comprising the at least one the active substance.

34. A method of treating or preventing a condition, disorder or a disease in a subject, the method comprising administering an effective amount of
i) at least one compound of Formula (I) 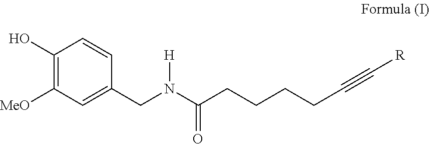
wherein R is alkyl, trifluoromethyl, cycloalkyl, phenyl, or halogen, and when the substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl, or a composition comprising the compound of Formula (I); and
ii) at least one active substance selected from the group consisting of Pirfenidone, Ramelteon, Terbinafine, Zolpidem, caffeine, melatonin, duloxetine and tizanidine, or tautomers or salts thereof,
to treat or prevent the condition, disorder or disease;
wherein the compound of Formula (I), acting as a bioenhancer of the at least one active substance, increases the bioavailability of the at least one active substance.

35. The method according to claim 34, wherein R is a C1-C6 alkyl or phenyl.

36. The method according to claim 34, wherein the at least one active substance is melatonin, and the condition, disorder or disease is a sleep condition, disorder or disease.

37. The method according to claim 34, wherein the at least one active substance is caffein, and the caffein is an energy supplement to restore mental alertness or wakefulness.

38. The method according to claim 34, wherein the method comprises oral administration.

39. A method for increasing the bioavailability of at least one active substance in a subject, wherein the at least one active substance is selected from the group consisting of Pirfenidone, Ramelteon, Terbinafine, Zolpidem, caffeine, melatonin, duloxetine and tizanidine, or tautomers or salts thereof,
the method comprising:
a step of administering a compound of Formula (I) 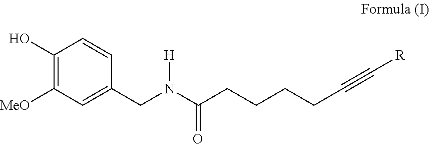
wherein R is alkyl, trifluoromethyl, cycloalkyl, phenyl, or halogen, when the substituent R comprises a carbon chain, it is straight-chained or branched and optionally further substituted with alkyl, alkenyl, alkynyl, allyl, aryl, alkoxy, aryloxy, alkanoyl, aroyl, amino alkylthio, arylthio, cyano, cycloalkyl, cycloalkenyl, halo, hydroxy, oxo, nitro, or trifluoromethyl,
to the subject,
wherein the compound acts as a bioenhancer of the at least one active substance.

40. The method of using a compound according to claim 23, wherein R is a C1-C4 alkyl.

41. The method of using a compound according to claim 40, wherein R is isopropyl.

42. The composition according to claim 31, wherein the at least one active substance is melatonin or caffeine.

43. The method according to claim 34, wherein R is a C1-C4 alkyl.

44. The method according to claim 43, wherein R is isopropyl.

45. The method according to claim 36, wherein the sleep condition, disorder or disease is sleep onset latency.
